(19) United States
(12) Patent Application Publication
(10) Pub. No.: US 2009/0149361 A1
(43) Pub. Date: Jun. 11, 2009

(54) Personal cleansing kit for promoting relaxation and sleep, reducing stress and decreasing muscle tightness

(76) Inventors: Emily Kathleen Adkison, Cincinnati, OH (US); June Turkanis Brennock, Loveland, OH (US); Victor Ruben Rodriguez, West Chester, OH (US)

Correspondence Address: THE PROCTER & GAMBLE COMPANY;Global Legal Department - IP, Sycamore Building - 4th Floor, 299 East Sixth Street, CINCINNATI, OH, 45202, US

(21) Appl. No.: 12/001,411

(22) Filed: Dec. 11, 2007

(51) Int. Cl. A61K 8/18; A61Q 19/00; A61Q 5/00

(52) U.S. Cl. 510/119; 510/130

(57) ABSTRACT

The present invention relates to a personal cleansing kit, that when used according to the instructions, provides a reduction in stress levels and muscle tightness, and improved sleep and relaxation. In one embodiment the personal cleansing kit comprises an article of commerce, a musical recording; and a heated cream. In another embodiment, the personal cleansing kit comprises an article of commerce an aroma releasing composition and a heated cream. In another embodiment, the personal cleansing kit comprises an article of commerce an aroma releasing composition, a musical recording and a heated cream. The article of commerce comprises a personal cleansing composition contained within a container and a set of instructions in association with said container. The set of instructions includes a breathing step, a stretching step and a massaging step.

### FIELD OF THE INVENTION

This invention relates to a personal cleansing kit that can be used for promoting relaxation and sleep, and reducing stress and decreasing muscle tightness.

### BACKGROUND OF THE INVENTION

Advances in technology in the last century have brought benefits to society but have resulted in greater prevalence of stress in the daily lives of people at all levels of society. It is known that stress can cause or aggravate many conditions including immunosuppression and vulnerability to infectious diseases, gastric conditions, sleep problems, depression, premature birth in expectant mothers, low birth weight, musculoskeletal problems, degeneration of brain neurons leading to memory and learning problems, elevated blood pressure, heart complications and stroke due to elevated blood lipid levels and other health complications.

The region in the brain known as the hypothalamus drives the activity of the mammalian stress response. Specifically, the hypothalamus drives the production of “stress hormones” including catecholamines and glucocorticoids. The hypothalamus responds to a stressor by activating the sympathetic nerve endings in the adrenal medulla to produce adrenaline. The hypothalamus produces corticotrophin-releasing hormone (“CRH”) which acts upon the pituitary to release adrenocorticotrophic hormone (“ACTH”) which in turn acts upon the adrenal cortex to promote the production of cortisol. The CRH and sympathetic systems participate in a positive feedback loop so that activation of one system activates the other. Since increased cortisol secretion is an indication that the HPA (“HPA”) axis has been activated, conversely, a decrease in cortisol secretion would indicate a downregulation of HPA axis activity.

While in the short term, the activation of these physiological responses to stress can have beneficial and even life saving merits; long-term or chronic stress has negative effects on health and well being. If the physiological response to chronic stress is to lead to elevated production of stress hormones, in effect resetting their basal levels, then it could be hypothesized that sustained reduction of these hormones, namely resetting the basal levels to a lower value, would be beneficial in managing stress and promoting well being. Also, as these hormones act upon each other in a positive feedback loop, downregulation of one system would be expected to downregulate the other. Resetting the basal levels of these stress hormones to a lower value could provide benefits including reduced perceived stress occurrence of other health and behavioral complications that are caused or aggravated by stress.

A good measure of the reactivity of the HPA axis is a measure of adrenocortical activity. An adrenocortical hormone that can be easily measured is cortisol, which can be found in the blood, urine and the saliva of human beings. Cortisol is produced in the adrenal cortex and is involved in a number of neurological events. Some have found that the level of this hormone rises when an individual is subjected to psychological and/or physiological stress. See Kirschbaum, C. & Hellhammer, D. H., “Salivary Cortisol in Psychoendocrine Research: Recent Developments and Applications”; Psychoendocrinology, Vol. 19 No. 4, 1994, pp. 313-333. Methodology to accurately measure this adrenocortical 110 (D hormone has been developed and refined over the past decade and is now applicable to measure HPA axis activity.

It has been recognized by those skilled in the art that a stressor induces an increase in the level of cortisol that is detectable in saliva. Reports of elevated salivary cortisol in response to psychological and physiological stress are reported by Kirschbaum, C. & Hellhammer, D. H., “Salivary Cortisol in Psychoendocrine Research: Recent Developments and Applications”; Psychoneuroendocrinology, Vol. 19 No. 4, 1994 pp. 313-333. Others have found that when adults are subjected to psychological stress (practicing arithmetic under stressful conditions) that their level of stress can be monitored by their salivary cortisol, see JP Patent No. 11-19076. The same researchers have shown that if the same individuals were exposed to certain fragrances before the stressful event, their level of salivary cortisol levels would not be as high as when they were psychologically challenged without the fragrance.

Many currently marketed fragrant cosmetic products claim a “calming”, “stimulating” or “relaxing” benefit to the user. Typically, these products possess fragrances that are purported to deliver these benefits. Measures of salivary cortisol have been used in this disclosure to demonstrate the downregulation of endocrine parameters in the stress response system and to relate this physiological downregulation to a reduction in perceived stress. This downregulation of the HPA axis, as measured by cortisol reduction, could be sufficient to reset basal levels.

It is further known that massage and music can be “calming”, “stimulating” or “relaxing” to the individual.

### SUMMARY OF THE INVENTION

The present invention relates to a personal cleansing kit, that when used according to the instructions, provides a reduction in stress levels and improved sleep and relaxation. In one embodiment the personal cleansing kit comprises an article of commerce, a musical recording, and a heated cream. In another embodiment, the personal cleansing kit comprises an article of commerce an aroma releasing composition and a heated cream. In another embodiment, the personal cleansing kit comprises an article of commerce an aroma releasing composition, a musical recording and a heated cream. The article of commerce comprises a personal cleansing composition contained within a container or a bar and a set of instructions in association with said container. The set of instructions includes a breathing step, a stretching step and a massaging step. The present invention gives the user a convenient way to have multiple relaxing products intended to work synergistically with one another, thereby providing the greatest amount of cortisol reduction.

### DETAILED DESCRIPTION OF THE INVENTION

### I. Definitions

The term “anhydrous” as used herein, unless otherwise specified, refers to those compositions or materials containing less than about 10%, more preferably less than about 5%, even more preferably less than about 3%, even more preferably zero percent, by weight of water.

The term “cleansing implement” as used herein is meant as the device, or instrument used in performing the task of cleansing. Suitable cleansing implements include but are not limited to cleansing puff, sponge, brush, wash cloth, disposable cloth, and the like.

The term “multiphase” as used herein means that compositions comprise at least two phases which are chemically distinct (e.g. a surfactant phase and a benefit phase). These phases are in direct physical contact with one another and are not separated by a barrier. In one aspect of the invention, phases of the multiphase personal care composition are blended or mixed to a significant degree. In another aspect of the invention, the phases of the multiphase personal care composition are made to occupy separate but distinct physical spaces inside the package in which they are stored, but are in direct contact with one another (i.e., they are not separated by a barrier and they are not emulsified or mixed to any significant degree). In one preferred embodiment of the present invention, the “multi-phase” personal care compositions comprise at least two visually distinct phases which are present within the package as a visually distinct pattern. The “patterns” or “patterned” include but are not limited to the following examples: striped, marbled, rectilinear, interrupted striped, check, mottled, veined, clustered, speckled, geometric, spotted, ribbons, helical, swirl, arrayed, variegated, textured, grooved, ridged, waved, sinusoidal, spiral, twisted, curved, cycle, streaks, striated, contoured, anisotropic, laced, weave or woven, basket weave, spotted, and tessellated. The ratio of a first phase to a second phase can be from about 90:10 to about 10:90, from about 80:20 to about 20:80, from about 70:30 to about 30:70, from about 60:40 to about 40:60, and about 50:50.

The term “personal care composition” as used herein, refers to compositions intended for topical application to the skin or hair. The compositions of the present invention are rinse-off compositions, in which the product is applied topically to the skin or hair and then is subsequently rinsed within minutes from the skin or hair with water, or otherwise wiped off using a substrate with deposition of a portion of the composition. The compositions also may be used as shaving aids. The personal care composition of the present invention is typically extrudable or dispensible from a package. The personal care compositions typically exhibit a viscosity of from about 1,500 centipoise (cP) to about 1,000,000 cP, as measured by as measured by the Viscosity Method as described in the commonly owned, patent application published on Nov. 11, 2004 under U.S. Publication No. 2004/0223991A1 by Wei, et al. The personal care compositions of the present invention can be in the form of liquid, semi-liquid, cream, lotion, bar, or gel compositions intended for topical application to skin. Examples of personal care compositions of the present invention can include but are not limited to shampoo, conditioning shampoo, body wash, moisturizing body wash, shower gels, skin cleansers, cleansing milks, hair and body wash, pet shampoo, shaving preparations and cleansing compositions used in conjunction with a disposable cleansing cloth.

A “skin compatible oil”, as defined herein, is an oil that is liquid or semi-solid at the temperature at which bathing is carried out that is deemed safe for use in cosmetics being either inert to the skin or actually beneficial. The most useful skin compatible oils for the present invention include ester oils, hydrocarbon oils, and silicone oils. Examples of these skin compatible oils are described in U.S. Publication No. 2004/0223992, U.S Publication No. 2003/0190296, U.S Publication No. 2003/0054019, U.S Publication No. 2003/0049282, U.S. Pat. No. 6,699,488 and U.S. Pat. No. 6,645,511.

The term “structured,” as used herein means having a rheology that confers stability on the composition. The degree of structure is determined by characteristics determined by one or more of the following methods the Yield Stress Method, or the Zero Shear Viscosity Method or by the Ultracentrifugation Method, all disclosed in co-pending U.S. application Ser. No. 11/312615 entitled “Shaving Kit, Article of Commerce and Method of Shaving Comprising a Personal Care Composition” filed Dec. 20, 2005. Accordingly, a surfactant phase of the personal care composition of the present invention is considered “structured,” if the surfactant phase has one or more of the following properties described below according to the Yield Stress Method, or the Zero Shear Viscosity Method or by the Ultracentrifugation Method. A surfactant phase is considered to be structured, if the phase has one or more of the following characteristics:

A. a Yield Stress of greater than about 0.1 Pascal (Pa), more preferably greater than about 0.5 Pa, even more preferably greater than about 1.0 Pa, still more preferably greater than about 2.0 Pa, still even more preferably greater than about 3 Pa, and even still even more preferably greater than about 5 Pa as measured by the Yield Stress and Zero Shear Viscosity Method described hereafter:

B. a Zero Shear Viscosity of at least about 500 Pascal-seconds (Pa-s), preferably at least about 1,000 Pa-s, more preferably at least about 1,500 Pa-s, even more preferably at least about 2,000 Pa-s; or

C. a Structured Domain Volume Ratio as measured by the Ultracentrifugation Method described hereafter, of greater than about 40%, preferably at least about 45%, more preferably at least about 50%, more preferably at least about 55%, more preferably at least about 60%, more preferably at least about 65%, more preferably at least about 70%, more preferably at least about 75%, more preferably at least about 80%, even more preferably at least about 85%; or

The phrase “substantially free of” as used herein, unless otherwise specified means that the composition comprises less than about 5%, less than about 3%, less than about 1% and less than about 0.1% of the stated ingredient. The term “free of” as used herein means that the composition comprises 0% of the stated ingredient which has not been added to the composition, however, these ingredients may incidentally form as a byproduct or a reaction product of the other components of the composition.

The term “surfactant component” as used herein means the total of all anionic, nonionic, amphoteric, zwitterionic and cationic surfactants in a phase. When calculations are based on the surfactant component, water and electrolyte are excluded from the calculations involving the surfactant component, since surfactants as manufactured typically are diluted and neutralized.

The term “visually distinct” as used herein, refers to a region of the personal care composition having one average composition, as distinct from another region having a different average composition, wherein the regions are visible to the unaided naked eye. This would not preclude the distinct regions from comprising two similar phases where one phase could comprise pigments, dyes, particles, glitter, pearlescence and various optional ingredients, hence a region of a different average composition. A phase generally occupies a space or spaces having dimensions larger than the colloidal or sub-colloidal components it comprises. A phase can also be constituted or re-constituted, collected, or separated into a bulk phase in order to observe its properties, e.g., by centrifugation, filtration or the like.

### II. Personal Cleansing Kit

The personal cleansing kit of the present invention can comprises a personal cleansing article, a musical recording and a heated cream. The personal cleansing kit of the present invention can comprise a personal cleansing article, an aroma releasing composition, and a heated cream. The personal cleansing kit of the present invention can comprise a personal cleansing article, an aroma releasing composition, a musical recording and a heated cream. The personal cleansing article comprises a container, a personal cleansing composition contained with a container and a set of instructions in association with the container. The personal cleansing article can comprise a soap bar or moisturizing soap bar with a wrapper and a set of instructions in association with the wrapper. The personal cleansing kit can comprise a second personal cleansing article, a third personal cleansing article, a fourth, etc. The personal cleansing composition can comprise shampoo, conditioning shampoo, conditioner, body wash, soap bar, moisturizing body wash, in-shower body moisturizer, shower gels, skin cleansers, cleansing milks, hair and body wash, shaving preparations and cleansing compositions used in conjunction with a disposable cleansing cloth.

The kit, first personal cleansing article, and second personal cleansing article can further comprise a coupon, rebate, or advertisement. The coupon, rebate or advertisement is associated with one or more of the personal cleansing products selected from the group consisting of a shampoo, conditioner, body lotion, skin care composition, deodorant, antiperspirant, after shave lotion, shaving razor, razor blade cartridges, and mixtures thereof. The kit can also comprise a grooming device selected from the group consisting of a cleansing cloth, disposable cleansing cloth (e.g. such as those described in U.S. Pat. Nos. 5,972,361; 5,980,931; 6,060,149; 6,063,397; 6,074,655; 6,132,746; 6,153,208; 6,280,757; 6,338,855; 6,495,151; 6,955,817), a pumice stone, a brush, a comb, a mirror, tweezers and mixtures thereof. The mirror can be a steam free, condensation proof, or non fogging mirror to facilitate shaving in the shower or bath. The components of the kit may be shrink-wrapped together or packaged together in a plastic package. The components of kit may be sold separately where each component's package enhances the idea of a kit, for example like in commonly owned and assigned U.S. Publication No. 2006/0280760A1 entitled “Assortment of personal care products having synergistic identifiers communicating products to be purchased together” and commonly owned and assigned U.S. Publication No. 2007/0100657A1 entitled “System of products having complimentary outer surface contours and indicia.”

### a. Personal Cleansing Article

The personal cleansing article or articles of the present invention comprise a container or package comprising a personal cleansing composition and instructions associated with the container or package. The personal cleansing article can comprise a soap bar, moisturizing soap, or high moisture bar with a wrapper and a set of instructions in association with the wrapper. Suitable soap bars are described in commonly owned and assigned U.S. patents including U.S. Pat. Nos. 5,300,249, 5,510,050, 5,681,980, 5,723,420, 6,080,707, 6,194,364 and U.S. application publication no. 2007/0155639A1 published on Jul. 5, 2007. The term “container” or “package” includes any suitable container for personal care compositions exhibiting a viscosity from about 1,500 centipoise (cP) to about 1,000,000 cP, including but not limited to a bottle, tottle, tube, jar, non-aerosol pump and mixtures thereof. As used herein “tottle” refers to a bottle which rests on the neck or mouth which its contents are filled in and dispensed from, but it is also the end upon which the bottle is intended to rest or sit upon for storage by the consumer and/or for display on the store shelf, as described in the commonly owned U.S. patent application Ser. No. 11/067443 filed on Feb. 25, 2005 to McCall, et al, entitled “Multi-phase Personal Care Compositions, Process for Making and Providing, and Article of Commerce.” The container can comprise a dispensing orifice and cap or lid. The container can be transparent, opaque or opalescent. The wrapper for the soap bar can be any known to those skilled in the art including a cardboard box, paperboard material, recycled paperboard, thermoplastic material, or a mixture thereof. The laminate material of the bar soap package comprises a paperboard material, a thermoplastic material disposed on at least one side of the paperboard material, and a fungicide. The paperboard material can be made of cellulosic fibers, either virgin pulp or recycled pulp. Recycled paperboard material is preferred. The thermoplastic material is preferably a biaxially oriented polypropylene material. The fungicide is preferably selected from the group consisting of carbendazim, chlorothalonil, p-tolyl diiodomethyl sulfone, thiabendazole, and mixtures thereof. Suitable soap bars and wrappers are disclosed in a commonly owned and assigned U.S. Patent application entitled “PACKAGING FOR HIGH MOISTURE BAR SOAP” filed on Dec. 1, 2006.

The instructions associated with the personal cleansing article instruct the consumer to undergo a series of steps during the cleansing time in the shower. The steps ask the consumers to use their bodies in a manner which causes an interaction with the personal care products and the shower. Through the instructions and interaction with the personal care composition, the consumers showering experience transforms from a daily routine step to a relaxation ritual. Importantly, the instruction are to communicate to the consumer the best way to treat their bodies, interact with the personal care composition and prepare for the additional components of the personal cleansing kit to promote a calm sanctuary in the shower. This instructions user to undergo three steps: a breathing step, a stretching step and a massaging step during the consumer's cleansing in the shower. The breathing step allows the consumer's olfactory senses to interact with the aromatic components of the personal care compositions and aroma releasing compositions, such as the essential oils, the volatile agents and perfuming elements. The breathing step instructs the consumer to inhale deeply and exhale slowly. The stretching step instructs the consumer to stretch by using slow and comfortable movements with their head. The massaging step instructs the consumer to massage their neck and shoulder in a circular motion. This step is enhanced by the addition a heated cream within the kit allows the consumers tacticle sensors to interact with the personal care composition. All of these steps are done within the shower during the time the consumer uses to cleanse in the shower. It is further understood that said instructions associated with the personal article may communicate the same actions using a different order or wording the phases differently. For example, another set of instructions associated with the personal cleansing article can instruct the user to: massage your shoulders and neck in a gradual motion, stretch by tilting your head in each direction and, continually take long and deep breaths as you use the product.

The personal cleansing composition comprises a cleansing phase. The personal cleansing composition can be multiphase and visually distinct. The structured surfactant phase comprises surfactants suitable for application to the skin or hair and which are otherwise compatible with the other essential ingredients in the personal care composition including water. These surfactants include anionic, nonionic, cationic, zwitterionic, amphoteric surfactants, soap, or combinations thereof. The personal care composition comprises from about 5% to about 16%, from about 10% to about 16%, from about 13% to about 15%, by weight of the personal care composition, of lathering surfactants selected from the group consisting of anionic surfactants, nonionic surfactants, amphoteric surfactants, cationic surfactants or mixtures thereof.

Suitable surfactants are described in McCutcheon's, Detergents and Emulsifiers, North American edition (1986), published by allured Publishing Corporation; and McCutcheon's, Functional Materials, North American Edition (1992); and in U.S. Pat. No. 3,929,678 issued to Laughlin, et al on Dec. 30, 1975.

Preferred linear anionic surfactants for use in the structured surfactant phase of the personal care composition include ammonium lauryl sulfate, ammonium laureth sulfate, sodium lauryl sulfate, sodium laureth sulfate, potassium laureth sulfate, sodium lauryl sarcosinate, sodium lauroyl sarcosinate, lauryl sarcosine, cocoyl sarcosine, ammonium cocoyl sulfate, potassium lauryl sulfate, and combinations thereof.

Branched anionic surfactants and monomethyl branched anionic surfactants suitable for the present invention are described in a commonly owned, patent application published on December, 2006 under U.S. Publication No. 60/680,149 by Smith, et al. Branched anionic surfactants include but are not limited to the following surfactants: sodium trideceth sulfate, sodium tridecyl sulfate, sodium C₁₂-₁₃ alkyl sulfate, and C₁₂-₁₃ pareth sulfate and sodium C₁₂-₁₃ pareth-n sulfate.

In one aspect, the personal care compositions of the present invention may further preferably comprise an amphoteric surfactant, a zwitterionic surfactant and mixtures thereof. In one embodiment, the personal care composition can comprise at least one amphoteric surfactant. Amphoteric surfactants suitable for use in the present invention include those that are broadly described as derivatives of aliphatic secondary and tertiary amines in which the aliphatic radical can be straight or branched chain and wherein one of the aliphatic substituents contains from about 8 to about 18 carbon atoms and one contains an anionic water solubilizing group, e.g., carboxy, sulfonate, sulfate, phosphate, or phosphonate. Examples of compounds falling within this definition are sodium 3-dodecyl-aminopropionate, sodium 3-dodecylaminopropane sulfonate, sodium lauryl sarcosinate, N-alkyltaurines such as the one prepared by reacting dodecylamine with sodium isethionate according to the teaching of U.S. Pat. No. 2,658,072, N-higher alkyl aspartic acids such as those produced according to the teaching of U.S. Pat. No. 2,438,091, and the products described in U.S. Pat. No. 2,528,378. In one aspect, the personal care composition can comprise an amphoteric surfactant that is selected from the group consisting of sodium lauroamphoacetate, sodium cocoamphoactetate, disodium lauroamphoacetate disodium cocodiamphoacetate, and mixtures thereof.

Zwitterionic surfactants suitable for use include those that are broadly described as derivatives of aliphatic quaternary ammonium, phosphonium, and sulfonium compounds, in which the aliphatic radicals can be straight or branched chain, and wherein one of the aliphatic substituents contains from about 8 to about 18 carbon atoms and one contains an anionic group, e.g., carboxy, sulfonate, sulfate, phosphate, or phosphonate. Zwitterionic surfactants suitable for use in the personal care composition include betaines, including cocoamidopropyl betaine.

In some aspects, the personal care composition of the present invention is preferably free of alkyl amines and alkanolamide to ensure mildness of the composition to the skin.

The personal care composition preferably comprises at least one nonionic emulsifier. Preferably the nonionic emulsifier has an HLB from about 1.5 to 13.0, preferably from about 3.4 to 13.0, more preferably 3.4 to about 9.5, more preferably 3.4 to about 8.0. The personal care composition preferably comprises a nonionic emulsifier at concentrations ranging from about 0.1% to about 10%, more preferably from about 0.25% to about 8%, even more preferably from about 0.5% to about 5%, still even more preferably from about 1.0% to about 3%, and still even still more preferably from about 1.5% to about 2.5%, by weight of the personal care compositions.

The balance between the hydrophilic and lipophilic moieties in a surfactant molecule is used as a method of classification (hydrophile-lipophile balance, HLB). The HLB values for commonly-used surfactants are readily available in the literature (e.g., HLB Index in McCutcheon's Emulsifiers and Detergents, MC Publishing Co., 2004). For example, cocamide monoethanolamine (CMEA) is known in the art to have an HLB value of 16.8. Another way of obtaining HLB values is to estimate by calculations. The HLB system was originally devised by Griffin (J. Soc. Cosmetic Chem., 1, 311, 1949). Griffin defined the HLB value of a surfactant as the mol % of the hydrophilic groups divided by 5, where a completely hydrophilic molecule (with no non-polar groups) had an HLB value of 20. Other examples of how to calculate HLB values are described by Davies in Interfacial Phenomena, 2nd Edition, Academic Press, London, 1963 and by Lin in J. Phys. Chem. 76, 2019-2013, 1972. Non-limiting examples of preferred nonionic emulsifiers for use herein are those selected form the group consisting of glyceryl monohydroxystearate, isosteareth-2, trideceth-3, hydroxystearic acid, propylene glycol stearate, PEG-2 stearate, sorbitan monostearate, glyceryl laurate, laureth-2, cocamide monoethanolamine, lauramide monoethanolamine, and mixtures thereof.

An electrolyte can be added per se to the personal care composition or it can be formed in situ via the counterions included in one of the raw materials. The electrolyte includes an anion that comprises phosphate, chloride, sulfate or citrate and a cation that comprises sodium, ammonium, potassium, magnesium or mixtures thereof. Some preferred electrolytes are sodium chloride, ammonium chloride, sodium or ammonium sulfate. The electrolyte is added to the structured surfactant phase in the amount, by weight of the personal care composition, of: from about 0.1% to about 6%, from about 1% to about 5%, from about 2% to about 4%, and from about 3% to about 4%.

The personal care compositions of the present invention can comprise a benefit phase. The benefit phase is comprised at least one benefit phase selected from the group consisting of a hydrophobic benefit phase, fatty compound gel network, a hydrophobic gel network, a hydrophobic gel network in a fatty compound gel network, a fatty compound gel network in a hydrophobic gel network, a silicone or silicone gel and mixtures thereof. Non-limiting examples of suitable benefit phase materials are disclosed in U.S. patent application Ser. No. 10/961,719.

The benefit phase in the present invention is preferably anhydrous and can be substantially free of water. The benefit phase can be substantially free or free of surfactant.

The benefit phase typically comprises hydrophobic moisturizing materials. The benefit phase may comprise from about 1% to about 50%, preferably from about 5% to about 30%, more preferably from about 10% to about 30%, by weight of the personal care composition, of a hydrophobic moisturizing material.

Hydrophobic moisturizing materials suitable for use in the present invention preferably have a Vaughan Solubility Parameter of from about 5 (cal/cm³)^{½} to about 15 (cal/cm³)^{½}, as defined by Vaughan in Cosmetics and Toiletries, Vol. 103. The Vaughan Solubility Parameter (VSP) as used herein is a parameter used to define the solubility of hydrophobic materials. Vaughan Solubility parameters are well known in the various chemical and formulation arts and typically have a range of from 5 to 25. Non-limiting examples of hydrophobic moisturizing materials having VSP values ranging from about 5 to about 15 include the following: Cyclomethicone 5.92, Squalene 6.03, Petrolatum 7.33, Isopropyl Palmitate 7.78, Isopropyl Myristate 8.02, Castor Oil 8.90, Cholesterol 9.55, as reported in Solubility, Effects in Product, Package, Penetration and Preservation, C. D. Vaughan, Cosmetics and Toiletries, Vol. 103, October 1988.

The hydrophobic moisturizing materials for use in the benefit phase of the composition have a preferred rheology profile as defined by Consistency value (k) and Shear Index (n). The term “Consistency value” or “k” as used herein is a measure of lipid viscosity and is used in combination with Shear Index, to define viscosity for materials whose viscosity is a function of shear. The measurements are made at 35° C. and the units are poise (equal to 100 cps). The term “Shear Index” or “n” as used herein is a measure of lipid viscosity and is used in combination with Consistency value, to define viscosity for materials whose viscosity is a function of shear. The measurements are made at 35° C. and the units are dimensionless. Consistency value (k) and Shear Index (n) are more fully described in co-pending U.S. application Ser. No. 11/312615 entitled “Shaving Kit, Article of Commerce and Method of Shaving Comprising a Personal Care Composition” filed Dec. 20, 2005. Preferred Consistency value ranges are 1-10,000 poise (1/sec)ⁿ⁻¹, preferably 10-2000 poise (1/sec)ⁿ⁻¹ and more preferably 50-1000 poise (1/sec)ⁿ⁻¹. Shear Index ranges are 0.1-0.8, preferably 0.1-0.5 and more preferably 0.20-0.4. These preferred rheological properties are especially useful in providing the personal cleansing compositions with improved deposition of benefit agents on skin.

The benefit phase can be comprised of the hydrophobic moisturizing materials selected from the group consisting of petrolatum, lanolin, derivatives of lanolin (e.g. lanolin oil, isopropyl lanolate, acetylated lanolin, acetylated lanolin alcohols, lanolin alcohol linoleate, lanolin alcohol riconoleate) hydrocarbon oils (e.g. mineral oil) natural and synthetic waxes (e.g. micro-crystalline waxes, paraffins, ozokerite, lanolin wax, lanolin alcohols, lanolin fatty acids, polyethylene, polybutene, polydecene, pentahydrosqualene) volatile or non-volatile organosiloxanes and their derivatives (e.g. dimethicones, cyclomethicones, alkyl siloxanes, polymethylsiloxanes, methylphenylpolysiloxanes), natural and synthetic triglycerides (e.g. castor oil, soy bean oil, sunflower seed oil, maleated soy bean oil, safflower oil, cotton seed oil, corn oil, walnut oil, peanut oil, olive oil, cod liver oil, almond oil, avocado oil, palm oil, sesame oil) and combinations thereof. In one aspect, at least about 50% by weight of the hydrophobic moisturizing materials are selected from the groups of petrolatum, mineral oil, paraffins, polyethylene, polybutene, polydecene, dimethicones, alkyl siloxanes, cyclomethicones, lanolin, lanolin oil, lanolin wax. The remainder of the hydrophobic moisturizing material can be selected from: isopropyl palmitate, cetyl riconoleate, octyl isononanoate, octyl palmitate, isocetyl stearate, hydroxylated milk glyceride and combinations thereof. The benefit phase of the personal care composition can be comprised a combination of petrolatum and mineral oil.

The in-shower body lotion compositions of the present invention are selected from the group consisting of skin compatibles oils, high internal phase emulsions, oil in water emulsions, gel networks, non-aqueous systems, and polyerically stabilized dispersions and mixtures thereof. Examples of in-shower body lotion compositions are described in detail in U.S. Publication No. 2004/0223992, U.S Publication No. 2003/0190296, U.S Publication No. 2003/0054019, U.S Publication No. 2003/0049282, U.S. Pat. No. 6,699,488 and U.S. Pat. No. 6,645,511. The personal cleansing composition can comprise one or more of an essential oil extracted a portion of a plant selected from the group consisting of whole flowers, petals, bark, leaves, twigs, whole fruit, fruit rind, seeds, roots, sap, and resins. The plant is selected from the group consisting of: acai, aloe vera, almond kernel, angelica, anise, apricot kernel, avocado, bamboo, basil, bay, benzoin, bergamot, birch, black pepper, bois de rose, borage, cajeput, camphor, carrot, cedar, chamomile, camomile mataricaria, chamomile roman, cinnamon, citronella, clary-sage, clove, coriander, corn, cumin, cypress, dill, eucalyptus, eucalyptus lemon, eucalyptus peppermint, eucalyptus radiata, evening primrose, fennel, frankincense, fungus, geranium, ginger, grapefruit, grapeseed, hazelnut, hops, hyssop, immortelle, inula odorata, jasmine, jojoba, juniper, kumquat, lavender, lemon, lemon grass, lime, mace, mandarin orange, majoram, melissa, mushroom, myrrh, neroli, niaouli, nutmeg, olive, orange, oregano, palma rose, parsley, patchouli, peach kernel, peanut, peppermint, petitgrain, pimento, pine, ravensara, red thyme, rose, rose bulger, rose maroc, rosemary, sage, sandalwood, sesame, sea cucumber, seaweed, soya, spearmint, sunflower, sweet almond, tagetes, tangerine, tea tree, thyme red, valerian, vetiver, violet, wheat germ, wintergreen, wild yam, yarrow, and ylang-ylang.

The personal cleansing composition can comprises a scent selected from the group consisting of lavender, violet, chamomile, ylang ylang, clary-sage, bergamot, frankincense, sandalwood, rose, bergamont, menthol, orange, peppermint, eucalyptus and mixtures thereof.

### b. Heated Cream

The personal cleansing kit can comprise a heated cream. The heated cream can be massaged into body parts during the shower experience. The heated cream can be in the form selected from the group consisting of semi-liquid, liquid, cream, gel, paste and mixtures thereof. Typically, the heated cream is self-heating or heated when activated by an exothermic reaction. The exothermic reaction can be the water of the shower. The heated cream can comprise an exothermic agent selected from the group consisting of aluminum bromide, aluminum chloride, aluminum iodide, aluminum sulfate hexahydrate, antimony pentachloride, barium hydroxide, barium iodide, barium oxide, barium oxide monohydrate, beryffium chloride, cadmium sulfate, calcium bromide, calcium chloride, calcium iodide, calcium oxide, cessium hydroxide, cessium oxide, chromium bromide hexahydrate, chromium chloride, hydrobromic acid, hydrochloric acid, hydriodic acid, ferrous chloride, ferric chloride, litium bromide, lithium iodide, lithium oxide, magnesium bromide, magnesium chloride, magnesium sulfate, magnesium iodide, managanese chloride, manganese sulfate, neodymium chloride, nickel nitrate, phosphorous trichloride, platinum chloride, potassium oxide, potassium sulfide, rubidium oxide, sodium tetraborate, sodium phosphate, sodium selenide, sodium sulfide, strontium bromide, stannic chloride, zinc chloride, zinc bromide, zinc sulfate, zinc iodide and combinations thereof.

### c. Aroma Releasing Composition

The personal cleansing kits of the present invention can comprise an aroma releasing composition. The aroma releasing composition provides a soothing aroma during the shower experience. In most embodiments, the fragrance from the aroma releasing composition is activated by the water of the shower. However, the aroma releasing composition can be activated by other means such as by a flame, when the aroma releasing composition is incense, or electricity, if the aroma releasing composition is in the form of a plug-in room santizer. The aroma releasing material can be places in a holder which can be releaseably attached to the shower wall, shower curtain or shower floor. The aroma releasing composition can be in the form selected from the group consisting of granular, tablet, suspension, oil, semi-liquid, liquid, cream, gel, paste and mixtures thereof. The aroma releasing composition may contain be comprised in a container. The aroma releasing composition can comprise a beta-cyclodextrin. The aroma releasing composition comprises a volatile agent selected from the group consisting of musk oil, civet, castreum, ambergris, plant perfumes, sandalwood oil, neroli oil, bergamot oil, lemon oil, lavender oil, sage oil, rosemary oil, peppermint oil, eucalyptus oil, menthol, camphor, verbena oil, citronella oil, cauout oil, salvia oil, clove oil, chamomille oil, sandalwood oil, costus oil, labdanum oil, broom extract, carrot seed extract, jasmine extract, minmosa extract, narcissus extract, olibanum extract, rose extract, acetophenonene, dimethylinadane derivatives, naphthaline derivatives, allyl caprate, α-amylcinnamic aldehyde, anethole, anisaldehyde, benzyl acetate, benzyl alcohol, benzyl propionate, borneol, cinnamyl acetate, cinnamyl alcohol, citral citronnellal, cumin aldehyde, cyclamen aldehyde, decanol, ethyl butyrate, ethyl caprate, ethyl cinnamate, ethyl vanillin, eugenol, geraniol, hexenol, α-hexylcinnamic aldehyde, hydroxycitrolnellal, indole, iso-amyl acetate, iso-amyl iso-valeratek iso-eugenol, linalol, linalyl acetate, p-methylacetophenone, methyl anthranilate, methyl dihydroasmonate, methyl eugenol, methyl-β-naphthol ketone, methylphenhlcarbinyl acetate, musk ketol, musk xylol, 2,5,6-nanodinol, γ-nanolactone, phenylacetoaldehydodimethyl acetate, β-phenylethyl alcohol, 3,3,5-trimethylcyclohexanol, γ-undecalactone, undecenal, vanillin and mixtures thereof.

An example of a suitable aromatic releasing composition can be found in U.S. Pat. No. 5,993,854 issued to Needlmen, et al.

### d. Musical Recording

The personal cleansing kit of the present invention can comprise a musical recording to provide relaxing auditory stimuli. Suitable auditory stimuli include, but are not limited to, music and sounds of nature that are soothing or relaxing to the consumer. The term music is used herein to include instrumental and lyrical compositions; tunes; melodies; harmonies; songs; beats and frequencies such as those from metronomes, hemisync, tuning forks, bells, beat machines, chimes; poetry, rhymes and mediations. The musical recording may be of any genre, including, but not limited to, the musical genre consisting of classical, soft rock, easy listening, jazz, progressive, county, alternative, alternative country, show tunes, nature sound, melodic, chimes, harp, and mixtures thereof. The sounds of nature include, but are not limited to, animal sounds, such as whales singing or birds chirping; insect sounds, such as crickets; and sounds of the environment, such as a running stream or a waterfall. Sounds that have consistently soft dynamics with minimal melodic and harmonic variability, having little or no conventional beat pitch, little or no vocal, slow tempo, little or no percussion or strong rhythm are particularly effective in relaxing or soothing the user. Sounds that use a binaural beat created by using two pure frequencies, usually one in each ear, are useful in improving the mood of the user. Binaural beats in the frequency range of delta, theta and alpha brain wave frequencies are useful for relaxing the user and beats in the frequency range of beta wave activity are useful for promoting mental alertness in the user. In one embodiment, the musical recording is selected according to brain waves studies.

The musical recording is selected from the form selected from a cassette tape, a compact disc, mp3, mpeg, wav, a computer chip, a computer audio file, downloadable music, and mixtures thereof. The musical recording could be in the form of computer chip which would be attached, integral, or associated with the container for the personal care composition or the kit itself. This computer chip may be activated by the consumer, such as by opening the lid of the personal care container, squeezing the container, dispensing the product, setting the contain down.

### Test Methods

### T-Bar Viscosity Method:

The viscosity of a composition contained in a zone can be assessed by the T-Bar Viscosity Method.

The apparatus for T-Bar measurement includes a Brookfield DV-II+Pro Viscometer with Helipath Accessory; chuck, weight and closer assembly for T-bar attachment; a T-bar Spindle D, a personal computer with Rheocalc software from Brookfield, and a cable connecting the Brookfield Viscometer to the computer. First, weigh 80 grams of the first or second composition in a 4-oz glass jar. Measure the T-bar viscosity by carefully dropping the T-Bar Spindle to the interior bottom of the jar and set the Helipath stand to travel in an upward direction. Open the Rheocalc software and set the following data acquisition parameters: set Speed to 5 rpm, set Time Wait for Torque to 00:01 (1 second), set Loop Start Count at 100. Start data acquisition and turn on the Helipath stand to travel upward at a speed of 22 mm/min. The T-Bar viscosity “T” is the average T-Bar viscosity reading between the 6^{th} reading and the 95^{th} reading (the first five and the last five readings are not used for the average T-Bar viscosity calculation). If the viscosity is below the lower limit of the D spindle (30,000 cps), a larger spindle can be used for the T-Bar Viscosity measurement.

### Ultracentrifugation Method:

The Ultracentrifugation Method is used to determine the percent of a structured domain or an opaque structured domain that is present in a multi-phase personal care composition that comprises a structured surfactant phase comprising a surfactant component. The method involves the separation of the composition by ultracentrifugation into separate but distinguishable layers. The multi-phase personal care composition of the present invention can have multiple distinguishable layers, for example a non-structured surfactant layer, a structured surfactant layer, and a benefit layer.

First, dispense about 4 grams of multi-phase personal care composition into Beckman Centrifuge Tube (11×60 mm). Next, place the centrifuge tubes in an Ultracentrifuge (Beckman Model L8-M or equivalent) and ultracentrifuge using the following conditions: 50,000 rpm, 18 hours, and 25° C.

After ultracentrifuging for 18 hours, determine the relative phase volume by measuring the height of each layer visually using an Electronic Digital Caliper (within 0.01 mm). First, the total height is measured as Hₐ which includes all materials in the ultracentrifuge tube. Second, the height of the benefit layer is measured as H_{b}. Third, the structured surfactant layer is measured as H_{c}. The benefit layer is determined by its low moisture content (less than 10% water as measured by Karl Fischer Titration). It generally presents at the top of the centrifuge tube. The total surfactant layer height (Hₛ) can be calculated by this equation:

Hₛ=Hₐ−H_{b}

The structured surfactant layer components may comprise several layers or a single layer. Upon ultracentrifugation, there is generally an isotropic layer at the bottom or next to the bottom of the ultracentrifuge tube. This clear isotropic layer typically represents the non-structured micellar surfactant layer. The layers above the isotropic phase generally comprise higher surfactant concentration with higher ordered structures (such as liquid crystals). These structured layers are sometimes opaque to naked eyes, or translucent, or clear. There is generally a distinct phase boundary between the structured layer and the non-structured isotropic layer. The physical nature of the structured surfactant layers can be determined through microscopy under polarized light. The structured surfactant layers typically exhibit distinctive texture under polarized light. Another method for characterizing the structured surfactant layer is to use X-ray diffraction technique. Structured surfactant layer display multiple lines that are often associated primarily with the long spacings of the liquid crystal structure. There may be several structured layers present, so that H_{c} is the sum of the individual structured layers. If a coacervate phase or any type of polymer-surfactant phase is present, it is considered a structured phase.

Finally, the structured domain volume ratio is calculated as follows:

Structured Domain Volume Ratio=H_{c}/Hₛ*100%

If there is no benefit phase present, use the total height as the surfactant layer height, Hₛ=Hₐ.

### Yield Stress and Zero Shear Viscosity Method:

The Yield Stress and Zero Shear viscosity of a composition contained in a zone can be assessed by the Yield Stress and Zero Shear Viscosity method.

A controlled stress rheometer such as a TA Instruments AR2000 Rheometer is used to determine the Yield Stress and Zero Shear Viscosity. The determination is performed at 25° C. with the 4 cm diameter parallel plate measuring system and a 1 mm gap. The geometry has a shear stress factor of 79580 m⁻³ to convert torque obtained to stress. Serrated plates can be used to obtain consistent results when slip occurs.

First a sample of the composition is obtained and placed in position on the rheometer base plate, the measurement geometry (upper plate) moving into position 1 mm above the base plate. Excess composition at the geometry edge is removed by scraping after locking the geometry. If the composition comprises particles discernible to the eye or by feel (beads, e.g.) which are larger than about 150 microns in number average diameter, the gap setting between the base plate and upper plate is increased to the smaller of 4 mm or 8-fold the diameter of the 95^{th} volume percentile particle diameter. If a composition has any particle larger than 5 mm in any dimension, the particles are removed prior to the measurement.

The determination is performed via the programmed application of a continuous shear stress ramp from 0.1 Pa to 1,000 Pa over a time interval of 4 minutes using a logarithmic progression, i.e., measurement points evenly spaced on a logarithmic scale. Thirty (30) measurement points per decade of stress increase are obtained. Stress, strain and viscosity are recorded. If the measurement result is incomplete, for example if material flows from the gap, results obtained are evaluated and incomplete data points excluded. The Yield Stress is determined as follows. Stress (Pa) and strain (unitless) data are transformed by taking their logarithms (base 10). Log(stress) is graphed vs. log(strain) for only the data obtained between a stress of 0.2 Pa and 2.0 Pa, about 30 points. If the viscosity at a stress of 1 Pa is less than 500 Pa-sec but greater than 75 Pa-sec, then log(stress) is graphed vs. log(strain) for only the data between 0.2 Pa and 1.0 Pa, and the following mathematical procedure is followed. If the viscosity at a stress of 1 Pa is less than 75 Pa-sec, the zero shear viscosity is the median of the 4 highest viscosity values (i.e., individual points) obtained in the test, the yield stress is zero, and the following mathematical procedure is not used. The mathematical procedure is as follows. A straight line least squares regression is performed on the results using the logarithmically transformed data in the indicated stress region, an equation being obtained of the form:

Log(strain)=m*Log(stress)+b (1)

Using the regression obtained, for each stress value (i.e., individual point) in the determination between 0.1 and 1,000 Pa, a predicted value of log(strain) is obtained using the coefficients m and b obtained, and the actual stress, using Equation (1). From the predicted log(strain), a predicted strain at each stress is obtained by taking the antilog (i.e., 10^{x} for each x). The predicted strain is compared to the actual strain at each measurement point to obtain a % variation at each point, using Equation (2).

% variation=100*(measured strain−predicted strain)/measured strain (2)

The Yield Stress is the first stress (Pa) at which % variation exceeds 10% and subsequent (higher) stresses result in even greater variation than 10% due to the onset of flow or deformation of the structure. The Zero Shear Viscosity is obtained by taking a first median value of viscosity in Pascal-seconds (Pa-sec) for viscosity data obtained between and including 0.1 Pa and the Yield Stress. After taking the first median viscosity, all viscosity values greater than 5-fold the first median value and less than 0.2× the median value are excluded, and a second median viscosity value is obtained of the same viscosity data, excluding the indicated data points. The second median viscosity so obtained is the Zero Shear Viscosity.

### EXAMPLES

Described below are examples of the components of the Personal Care Kit of the Present Invention:

Preparation of Personal Care Composition with Sensory Fragrance: Two compositions were made with two fragrances. Two compositions having two different fragrance formulations were prepared according to the formulas in Table 1. The compositions were prepared by conventional mixing techniques in the order of addition indicated. Addition step 8 in Table 1 contains water and Polyquatemium-10 that was premixed prior to addition to the batch.

TABLE 1
Compositions A and B comprising different fragrances

| | A | B |
|---|---|---|
| 1. Sodium Laureth 3-Sulfate | 9.1 | 9.1 |
| 2. Sodium Sulfate | 3.6 | 3.6 |
| 3. Cocamidopropyl Betaine | 1.43 | 1.43 |
| 4. Sodium Lauroamphoacetate | 0.95 | 0.95 |
| 5. Citric Acid | 0.3 | 0.3 |
| 6. Disodium EDTA | 0.12 | 0.12 |
| 7. Sodium Benzoate | 0.24 | 0.24 |
| 8. Water | QS | QS |
| Polyquaternium-10 | 0.24 | 0.24 |
| 9. Lavender Fragrance Formulation¹ | 2.0 | — |
| 9. Aloe Fragrance Formulation² | — | 1.0 |
| 10. Methyl Chloro Isothiazolinone & | 0.0005 | 0.0005 |
| Methyl Isothiazolinone | | |

¹“Lavender Silhouette WS 29087” purchased from Wessel Fragrance Inc, available from Wessel Fragrance, Inc located on 400 Sylvan Avenue, Engle wood Cliffs, NJ 07632 (Phone: 1-201-541-1119).
²“Perfume Hydro Aloe Bw Av (UI032942/00)” purchased from Givaudan Fragrances, available from Givaudan Fragrances located on 1775 Windsor Road, Teaneck.

Example of Heated Cream: Two compositions were made to create the heated cream. The two compositions are Example 1 and a heated formula. The heated formula was prepared according to the formula in Table 2. The composition was prepared by mix ingredients using conventional mixing techniques in the order of addition. Mix group A and heat mixture. Add ingredients in group B to group A and maintain heat until all ingredients are melted (˜80° C.). Then, add ingredient C. Cool to 45° C. Finally, add ingredients from group D and cool to 35° C.

TABLE 2
Compositions C with Lavender Oil

| Mixing | | |
|---|---|---|
| Groups | Ingredient | Composition C |
| A | PEG-4 | QS |
| A | Dioleoylethyl Hydroxyethylmonium | 1.00 |
| | Methosulfate | |
| A | Hydroxypropylcellulose | 0.25 |
| B | PEG/PPG = 300/55 Copolymer | 1.5 |
| B | Behenyltrimethylammonium Chloride | 0.5 |
| B | Stearyl Alcohol | 1.00 |
| B | Cetyl Alcohol | 0.5 |
| B | Distearyldimonium Chloride | 0.4 |
| B | Methylparaben | 0.2 |
| B | Propylparaben | 0.1 |
| C | Magnesium Sulfate | 20 |
| D | Polyethylene Beads | 5 |
| D | Lavender Oil Fragrance¹ | As per preference |

¹“Oil Lavender Bulgarian Pure FCC” purchased from Citrus and Allied Essences Ltd, available from Citrus and Allied Essences Ltd at http://citrusandallied.com.

The two compositions Example 1 and a heated formulas from Table 2 were packaged in 10 ml syringes, using BD 10 ml slip-tip disposable syringes available from BD with its reference number as 301604. Syringes were packed according to Table 3. Syringe 1 and 2 were taped together. During use, Syringe 1 and 2 were dispensed simultaneously. The total amount dispensed was 15 mL.

TABLE 3
Syringe Packaging Instructions

| | Syringe 1 | Syringe 2 |
|---|---|---|
| Fill Amount | 10 mL | 5 mL |
| Composition A | X | — |
| Composition C | — | X |
| Composition B | X | — |
| Composition C | — | X |

Example of Aroma Releasing Composition: Aroma Releasing has the composition of Example 1. The Aroma Releasing Composition was packaged into 2-20 mL syringes, using BD 20-mL slip-tip disposable syringes available from BD with its reference number as 301625. Syringes were packed according to Table 4. Syringes 1 and 2 were taped together. During use, syringes 1 and 2 were dispensed simultaneously. The total amount of 30 mL was dispensed.

TABLE 4
Syringe Packaging Instructions

| | Syringe 1 | Syringe 2 |
|---|---|---|
| Fill Amount | 10 mL | 20 mL |
| Composition A | X | — |
| Composition A | — | X |
| Composition A | X | — |
| Composition B | — | X |

During use, the syringes were either dispensed on the shower or tub floor, or dispensed into a wire screen cup and then placed on the shower or tube floor. More specifically, the dispensed location is where some of the shower's water hits Example 1 and activated the aroma releasing composition. Syringes were dispensed before the shower and immediately before the shower water was turned on.

Example of Application Instructions: Application instructions were provided with the personal care kits. They included order of products, how to apply products, deep breathing, stretching & music.

Relaxation Instructions (Welcome to your relaxing shower experience; this shower is designed to soothe your body, mind & soul.)

Step 1. Before you begin your shower, prepare your floor bloom. (To prepare the floor bloom: place the wire screen on the shower floor where water can lightly hit it; dispense the floor bloom syringes into the provided screen; turn on the water & adjust the water temperature to your liking.)

Step 2. When you're ready, enter the shower. Breathe in deeply & exhale slowly . . . savor the relaxing scents.

Step 3. Using the provided massage cream, dispense the cream into your hand. Apply the cream to you neck & shoulders in a circular motion. Breathe in deeply & exhale slowly . . . savor the relaxing scents.

Step 4. Using your shampoo, wash your hair & massage your head. Breathe in deeply & exhale slowly . . . savor the relaxing scents.

Step 5. Using the provided bodywash & puff, clean your body. Breathe in deeply & exhale slowly . . . savor the relaxing scents.

Step 6. Stand underneath the shower's water; let if flow over your back & neck. Breathe in deeply & exhale slowly . . . savor the relaxing scents.

Step 7. Stretching: Slowly & comfortably turn your head side to side (repeat 6 times). Breathe in deeply & exhale slowly . . . savor the relaxing scents.

Step 8. Stretching: Slowly & comfortably turn your head back & forth (repeat 6 times).

Step 9. Stand underneath the shower's water; let it flow over your back & neck. Breathe in deeply & exhale slowly . . . savor the relaxing scents.

When you are ready & through with your relaxing shower, ease from your shower as a soothed, relaxed person.

Saliva Collection and Cortisol Measurement: Described below is the testing of an embodiment of the personal care kit of the present invention. The testing included sampling and collection of participants' saliva for various shower conditions. Afterwards, the saliva was tested for the participants' cortisol levels.

Shower Kit and Saliva Collection 1: A total of nine females having an age of about 25 to 54 years, who regularly use bodywash and have multiple personal cleansing products in their respective shower, participated in a shower study in which their respective saliva samples were collected before and two points after showering for the purpose of measuring salivary cortisol concentrations. All showers were taken about 90 minutes after waking up.

Forty-five minutes prior to showering about 1.8 mL of an initial saliva sample was collected from each respective female following the Saliva Collection Method and Passive Drooling Method.

Saliva Collection Method: Saliva is collected by the Passive Drooling Method and stored a 1.8 mL polypropylene vial. About 1.8 mL of saliva was collected in the said vial. Saliva samples were frozen at −20C or lower until subsequently cortisol concentration analyses.

Passive Drooling Method: Ten minutes prior to collecting the saliva samples, panelists rinsed their mouth with water. Following the water rinse, all food, drinks, candy, medicines were avoided until after the saliva was collected. At the saliva collection time, panelists promoted saliva production, if necessary, by imaging eating their favorite food and/or moving their jaw in a chewing motion. Panelists would tilt their head forward and have the saliva pool behind their front teeth. Once sufficient saliva was in their mouth, panelists would place a drinking straw, about 2 inches long, on their lips and keeping their head tilted forward would allow the saliva to flow from their mouth and through the straw. The straw emptied into the 1.8 mL polypropylene vial. Panelists repeated the passive drooling method until about 1.8 mL of saliva was collected.

Each female was then asked to bathe independently in a shower. Example 1, 2, 3 and 4 were used during the shower.

Fifteen and forty-five minutes after each female had finished showering about 1.8 mL of post-showering saliva samples were collected from each female following the Saliva Collection Method and Passive Drooling Method. This second & third sample were also frozen for subsequent cortisol concentration analysis.

Each participant repeated these step five times on consecutive days.

Hot Water Shower and Saliva Collection 2: A total of nine females having an age of about 25 to 54 years, who regularly use bodywash and have multiple personal cleansing products in their respective shower, participated in a shower study. The same said females from Example 5. Their respective saliva samples were collected following the said Saliva Collection Method and Passive Drooling Method at the said times of 45 minutes before the shower and fifteen and forty-five minutes after showering for the purpose of measuring salivary cortisol concentrations. Each female repeated the procedure set forth in Example 5, with the exception panelists stood under the shower and used no products, neither test products nor their typical showering products. Each participant repeated these step four times on consecutive days.

Body Wash Shower and Saliva Collection 3: A total of nine females having an age of about 25 to 54 years, who regularly use bodywash and have multiple personal cleansing products in their respective shower, participated in a shower study. The same said females from Example 5. Their respective saliva samples were collected following the said Saliva Collection Method and Passive Drooling Method at the said times of 45 minutes before the shower and fifteen and forty-five minutes after showering for the purpose of measuring salivary cortisol concentrations. Each female repeated the procedure set forth in Example 5, with the exception that the females used Example 1 during the shower. Each participant repeated these step three times on consecutive days.

Salivary Cortisol Testing: Saliva samples obtained from the studies for the Shower Kit shower, the Hot Water Shower, and Body Wash Shower described above were tested for cortisol concentrations by Salimetrics, LLC using a “Salimetrics Expanded Range High Sensitivity Salivary Cortisol Enzyme Immunoassay Kit” available from Salimetrics, LLC in its catalog as “Catalog No. 1-3002/1-3012, 96-Well Kit” in accordance with the instructions contained therein. More specifically, the saliva samples were assayed for salivary cortisol in duplicate. The test uses 25 ul of saliva per determination, has a lower limit of sensitivity of 0.003 ug/dl, standard curve range from 0.012 to 3.0 ug/dl, and average intra-and inter-assay coefficients of variation 3.5% and 5.1% respectively. Method accuracy, determined by spike and recovery, and linearity, determined by serial dilution are 100.8% and 91.7%. Values from matched serum and saliva samples show the expected strong linear relationship, r (63)=0.89, p<0.0001.

The results of the cortisol analyses for saliva collection samples 1, 2 and 3. Cortisol data was analyzed by an analysis of covariance and are reported in Table 5. The significances (p-values for mean pairwise comparison) between saliva collection samples 1, 2 and 3 are reported in Table 6 & 7.

TABLE 5

| | | | |
|---|---|---|---|
| | | After | After |
| Panelist # | Before | (15 min) | (45 min) |

(A) Cortisol Results from saliva collection sample 1
(Shower Kit)

| | | | |
|---|---|---|---|
| 1 | 0.381 | 0.192 | 0.155 |
| 2 | 0.379 | 0.189 | 0.216 |
| 3 | 0.596 | 0.345 | 0.234 |
| 4 | 0.263 | 0.083 | 0.065 |
| 5 | 0.458 | 0.263 | 0.241 |
| 6 | 0.476 | 0.178 | 0.204 |
| 7 | 0.224 | 0.212 | 0.225 |
| 8 | 0.338 | 0.181 | 0.195 |
| 9 | 0.598 | 0.437 | 0.434 |
| Average | 0.412 | 0.231 | 0.219 |

(B) Cortisol Results from saliva collection sample 2
(Hot Water Shower)

| | | | |
|---|---|---|---|
| 1 | 0.496 | 0.264 | 0.280 |
| 2 | 0.288 | 0.254 | 0.183 |
| 3 | 0.907 | 0.390 | 0.301 |
| 4 | 0.338 | 0.126 | 0.093 |
| 5 | 0.734 | 0.629 | 0.564 |
| 6 | 0.625 | 0.414 | 0.323 |
| 7 | 0.190 | 0.252 | 0.230 |
| 8 | 0.442 | 0.209 | 0.218 |
| 9 | 0.446 | 0.533 | 0.499 |
| Average | 0.496 | 0.341 | 0.299 |

(C) Cortisol Results from saliva collection sample 3
(Body Wash Shower)

| | | | |
|---|---|---|---|
| 1 | 0.317 | 0.129 | 0.113 |
| 2 | 0.215 | 0.149 | 0.119 |
| 3 | 0.521 | 0.268 | 0.242 |
| 4 | 0.468 | 0.206 | 0.167 |
| 5 | 0.529 | 0.437 | 0.343 |
| 6 | 0.650 | 0.338 | 0.234 |
| 7 | 0.424 | 0.453 | 0.372 |
| 8 | 0.284 | 0.117 | 0.132 |
| 9 | 0.504 | 0.321 | 0.351 |
| Average | 0.435 | 0.269 | 0.230 |

TABLE 6
Signifances between saliva collection samples 1, 2 and 3
(After 15 min.)

| | Saliva | Salvia | |
|---|---|---|---|
| | Sample 1. | Sample 3 | Saliva Sample 2 |
| | (Shower Kit) | (Body Wash) | (Hot Water Shower) |
| Saliva Sample 1. | — | 0.09 | 0.01 |
| (Shower Kit) | | | |
| Salvia Sample 3 | — | — | 0.17 |
| (Body Wash) | | | |
| Saliva Sample 2 | — | — | — |
| (Hot Water Shower) | | | |

TABLE 7
Signifances between saliva collection samples 1, 2 and 3
(After 45 min.)

| | Saliva | Salvia | |
|---|---|---|---|
| | Sample 1. | Sample 3 | Saliva Sample 2 |
| | (Shower Kit) | (Body Wash) | (Hot Water Shower) |
| Saliva Sample 1. | — | 0.42 | 0.02 |
| (Shower Kit) | | | |
| Salvia Sample 3 | — | — | 0.10 |
| (Body Wash) | | | |
| Saliva Sample 2 | — | — | — |
| (Hot Water Shower) | | | |

This Example showed that the Saliva Sample 1 (Shower Kit) and Salvia Sample 3 (Body Wash) were effective at reducing cortisol levels in females. At 15 minutes after showering, Saliva Sample 1 (Shower Kit) is significantly better at reducing cortisol in females versus Saliva Sample 2 (Hot Water Shower) and Salvia Sample 3 (Body Wash) (90% confidence). At 45 minutes after showering, Saliva Sample 1 (Shower Kit) and Salvia Sample 3 (Body Wash) are significantly better at reducing cortisol levels in females versus Saliva Sample 2 (Hot Water Shower) (90% confidence).

Each of the panelists who participated in the salvia collection study were also given questionnaires regarding how the felt before and after each study shower. More specifically, the pre-shower & post-shower questionnaire addressed their current stress level & muscle tightness and/or tension on a 0-100 scale with 0 being no stress or no muscle tightness and 100 being extremely stressed or extreme muscle tightness. The results of the questionnaires are shown in Table 8 and 9.

TABLE 8
Stress Level from Questionnaires

| | Average Rating | Average Rating | |
|---|---|---|---|
| | for Pre-Shower | for Post-Shower | Delta |
| Saliva Sample 1. (Shower Kit) | 34.7 | 24.0 | −10.7 |
| (Average) | | | |
| Saliva Sample 2 (Hot Water | 34.7 | 28.6 | −6.0 |
| Shower) (Average) | | | |
| Salvia Sample 3 (Body Wash) | 33.9 | 28.8 | −5.0 |
| (Average) | | | |

TABLE 9
Muscle Tightness from Questionnaires

| | Average Rating | Average Rating | |
|---|---|---|---|
| | for Pre-Shower | for Post-Shower | Delta |
| Saliva Sample 1. (Shower Kit) | 33.9 | 21.9 | −12.0 |
| (Average) | | | |
| Saliva Sample 2 (Hot Water | 26.0 | 22.4 | −3.6 |
| Shower) (Average) | | | |
| Salvia Sample 3 (Body Wash) | 37.0 | 29.6 | −7.3 |
| (Average) | | | |

This example showed that the Shower Kit reduced stressed and reduced muscle tightness more than the Hot Water Shower and the Body Wash Shower and statistically significance (one-tailed paired t-test at 90% confidence). The Body Wash Shower had less muscle tightness than the Hot Water Shower and is statistically significant (one-tailed paired t-test at 90% confidence).

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

All documents cited in the Detailed Description of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A personal cleansing kit comprising:
a. an article of commerce comprising:
i. a personal cleansing composition contained with a container;
ii. a set of instructions in association with said container:
b. a musical recording; and:
c. a heated cream.

2. The personal cleansing kit according to claim 1, wherein said set of instructions further comprise the following steps:
1. a breathing step instructing the consumer to inhale deeply and exhale slowly;
2. a stretching step instructing the consumer to slowly and comfortably move their head; and
3. a massaging step instructing the consumer to massage their neck and shoulders in a circular motion.

3. The personal cleansing kit according to claim 1, wherein said personal cleansing composition is in the form selected from the group consisting of semi-liquid, liquid, cream, gel, paste, foam, mousse, and mixtures thereof.

4. The personal cleansing composition of claim 1, is selected from the group consisting of shampoo, conditioning shampoo, conditioner, body wash, moisturizing body wash, in-shower body moisturizer, shower gels, skin cleansers, cleansing milks, hair and body wash, shaving preparations and cleansing compositions used in conjunction with a disposable cleansing cloth.

5. The personal cleansing kit according to claim 1, wherein said personal cleansing composition comprises a hydrophobic moisturizing material is selected from the group consisting of petrolatum, mineral oil micro-crystalline waxes, paraffins, ozokerite, polyethylene, polybutene, polydecene and perhydrosqualene, dimethicones, cyclomethicones, alkyl siloxanes, polymethylsiloxanes and methylphenylpolysiloxanes, lanolin, lanolin oil, lanolin wax, lanolin alcohols, lanolin fatty acids, isopropyl lanolate, acetylated lanolin, acetylated lanolin alcohols, lanolin alcohol linoleate, lanolin alcohol riconoleate castor oil, soy bean oil, sunflower seed oil, maleated soy bean oil, safflower oil, cotton seed oil, corn oil, walnut oil, peanut oil, olive oil, cod liver oil, almond oil, avocado oil, palm oil and sesame oil, and mixtures thereof.

6. The personal cleansing kit according to claim 1, wherein said personal cleansing composition comprises one or more of an essential oil extracted a portion of a plant selected from the group consisting of whole flowers, petals, bark, leaves, twigs, whole fruit, fruit rind, seeds, roots, sap, and resins.

7. The personal cleansing kit according to claim 6, wherein the plant is selected from the group consisting of: acai, aloe vera, almond kernel, angelica, anise, apricot kernel, avocado, bamboo, basil, bay, benzoin, bergamot, birch, black pepper, bois de rose, borage, cajeput, camphor, carrot, cedar, chamomile, camomile mataricaria, chamomile roman, cinnamon, citronella, clary-sage, clove, coriander, corn, cumin, cypress, dill, eucalyptus, eucalyptus lemon, eucalyptus peppermint, eucalyptus radiata, evening primrose, fennel, frankincense, fungus, geranium, ginger, grapefruit, grapeseed, hazelnut, hops, hyssop, immortelle, inula odorata, jasmine, jojoba, juniper, kumquat, lavender, lemon, lemon grass, lime, mace, mandarin orange, majoram, melissa, mushroom, myrrh, neroli, niaouli, nutmeg, olive, orange, oregano, palma rose, parsley, patchouli, peach kernel, peanut, peppermint, petitgrain, pimento, pine, ravensara, red thyme, rose, rose bulger, rose maroc, rosemary, sage, sandalwood, sesame, sea cucumber, seaweed, soya, spearmint, sunflower, sweet almond, tagetes, tangerine, tea tree, thyme red, valerian, vetiver, violet, wheat germ, wintergreen, wild yam, yarrow, and ylang-ylang.

8. The personal cleansing kit according to claim 1, wherein said personal cleansing composition comprises a scent selected from the group consisting of lavender, violet, chamomile, ylang ylang, clary-sage, bergamot, menthol, frankincense, sandalwood, rose, bergamont, orange, peppermint, eucalyptus and mixtures thereof.

9. The personal cleansing kit according to claim 1, wherein said heated cream is in the form selected from the group consisting of semi-liquid, liquid, cream, gel, paste and mixtures thereof.

10. The personal cleansing kit according to claim 9, wherein said heated cream comprises an exothermic agent selected from the group consisting of aluminum bromide, aluminum chloride, aluminum iodide, aluminum sulfate hexahydrate, antimony pentachloride, barium hydroxide, barium iodide, barium oxide, barium oxide monohydrate, beryffium chloride, cadmium sulfate, calcium bromide, calcium chloride, calcium iodide, calcium oxide, cessium hydroxide, cessium oxide, chromium bromide hexahydrate, chromium chloride, hydrobromic acid, hydrochloric acid, hydriodic acid, ferrous chloride, ferric chloride, litium bromide, lithium iodide, lithium oxide, magnesium bromide, magnesium chloride, magnesium sulfate, magnesium iodide, managanese chloride, manganese sulfate, neodymium chloride, nickel nitrate, phosphorous trichloride, platinum chloride, potassium oxide, potassium sulfide, rubidium oxide, sodium tetraborate, sodium phosphate, sodium selenide, sodium sulfide, strontium bromide, stannic chloride, zinc chloride, zinc bromide, zinc sulfate, zinc iodide and combinations thereof.

11. The personal cleansing kit according to claim 1, wherein said musical recording is selected from the musical genre consisting of classical, soft rock, easy listening, jazz, progressive, county, alternative, alternative country, show tunes, nature sound, melodic, chimes, harp, and mixtures thereof.

12. The personal cleansing kit according to claim 1, wherein said musical recording is selected according to brain waves studies.

13. The personal cleansing kit according to claim 1, wherein said musical recording is selected from the form selected from a cassette tape, a compact disc, mp3, mpeg, wav, a computer chip, a computer audio file, downloadable music, and mixtures thereof.

14. The personal cleansing kit according to claim 1, further comprising an aroma releasing composition.

15. The personal cleansing kit according to claim 1, wherein said aroma releasing composition comprises a betacyclodextrin.

16. The personal cleansing kit according to claim 12, wherein said aroma releasing composition is in the form selected from the group consisting of granular, tablet, suspension, oil, semi-liquid, liquid, cream, gel, paste and mixtures thereof. and may contain a container to put the aroma releasing composition in.

17. The personal cleansing kit according to claim 12, wherein said aroma releasing composition comprises a volatile agent is selected from the group consisting of musk oil, civet, castreum, ambergris, plant perfumes, sandalwood oil, neroli oil, bergamot oil, lemon oil, lavender oil, sage oil, rosemary oil, peppermint oil, eucalyptus oil, menthol, camphor, verbena oil, citronella oil, cauout oil, salvia oil, clove oil, chamomille oil, sandalwood oil, costus oil, labdanum oil, broom extract, carrot seed extract, jasmine extract, minmosa extract, narcissus extract, olibanum extract, rose extract, acetophenonene, dimethylinadane derivatives, naphthaline derivatives, allyl caprate, α-amylcinnamic aldehyde, anethole, anisaldehyde, benzyl acetate, benzyl alcohol, benzyl propionate, borneol, cinnamyl acetate, cinnamyl alcohol, citral citronnellal, cumin aldehyde, cyclamen aldehyde, decanol, ethyl butyrate, ethyl caprate, ethyl cinnamate, ethyl vanillin, eugenol, geraniol, hexenol, α-hexylcinnamic aldehyde, hydroxycitrolnellal, indole, iso-amyl acetate, iso-amyl iso-valeratek iso-eugenol, linalol, linalyl acetate, p-methylacetophenone, methyl anthranilate, methyl dihydroasmonate, methyl eugenol, methyl-β-naphthol ketone, methylphenhlcarbinyl acetate, musk ketol, musk xylol, 2,5,6-nanodinol, γ-nanolactone, phenylacetoaldehydodimethyl acetate, β-phenylethyl alcohol, 3,3,5-trimethylcyclohexanol, γ-undecalactone, undecenal, vanillin and mixtures thereof.

18. The personal cleansing kit according to claim 1, wherein said personal cleansing composition is a body wash.

19. The personal cleansing kit according to claim 1, wherein said personal cleansing composition is a shampoo.

20. The personal cleansing kit according to claim 1, further comprising a second article of commerce further comprising a second personal cleansing composition.

21. The personal cleansing kit according to claim 18, wherein said second personal cleansing composition is selected from the group consisting of shampoo, conditioning shampoo, conditioner, body wash, moisturizing body wash, in-shower body moisturizer, shower gels, skin cleansers, cleansing milks, hair and body wash, shaving preparations and cleansing compositions used in conjunction with a disposable cleansing cloth.

22. The personal cleansing kit according to claim 1, wherein said kit further comprises a set of instructions associated with said kit.

23. A personal cleansing kit comprising:
a. an article of commerce comprising:
i. a personal cleansing composition contained with a container;
ii. a set of instructions in association with said container;
b. an aroma releasing composition; and;
c. a heated cream.

24. A personal cleansing kit comprising:
a. an article of commerce comprising:
i. a soap bar contained with a wrapper;
ii. a set of instructions in association with said wrapper;
b. a musical recording; and;
c. a heated cream.

25. A personal cleansing kit according to claim 1, wherein said kit further comprises a cleansing implement.
